(19) United States
(12) Patent Application Publication
(10) Pub. No.: US 2010/0234221 A1
(43) Pub. Date: Sep. 16, 2010

(54) USE OF TETRAMIC ACID DERIVATIVES WITH FERTILIZERS

(75) Inventors: Wolfram Andersch, Bergisch Gladbach (DE); Reiner Hungenberg, Langenfeld (DE); Peter Marczok, Koln (DE); Rolf Pontzen, Leichlingen (DE); Udo Reckmann, Koln (DE); Marie Alain Xavier Waetermeulen, Dusseldorf (DE); Jürgen Kühnhold, Bergisch Gladbach (DE); John Bell, Chapel Hill, NC (US); Stephen Krüger, Raleigh, NC (US); John Hinz, Story City, IA (US)

Correspondence Address: Baker Donelson Bearman, Caldwell & Berkowitz, PC, 920 Massachusetts Ave, NW, Suite 900, Washington, DC, 20001, US

(73) Assignee: BAYER CROPSCIENCE AG, Monheim (DE)

(21) Appl. No.: 12/376,411

(22) PCT Filed: Jul. 27, 2007
(86) PCT No.: PCT/EP2007/006649

§ 371 Date: Jun. 18, 2009

(30) Foreign Application Priority Data
Aug. 9, 2006 (EP) 06016607.1

(51) Int. Cl. C05G 3/02; C07D 209/54

(52) U.S. Cl. 504/101; 548/408

(57) ABSTRACT

The present invention relates to boosting the activity of crop protection compositions comprising phenyl-substituted tetramic acid derivatives by adding fertilizers, and to corresponding compositions.

The present invention relates to boosting the activity of crop protection compositions comprising phenyl-substituted tetramic acid derivatives by adding fertilizers, to the corresponding compositions and to their use in crop protection.

The insecticidal and acaricidal activity of tetramic acid derivatives following spray application is disclosed in EP-A-456 063, EP-A-521 334, EP-A-596 298, EP-A-613 884, WO 95/01 997, WO 95/26 954, WO 95/20 572, EP-A-0 668 267, WO 96/25 395, WO 96/35 664, WO 97/01 535, WO 97/02 243, WO 97/36 868, WO 97/43 275, WO 98/05638, WO 98/06721, WO 98/25928, WO 99/16748, WO 99/24437, WO 99/43649, WO 99/48869, WO 99/55673, WO 01/09092, WO 01/17972, WO 01/23354, WO 01/74770, WO 02/19824, WO 03/013249, WO 04/007 448, WO 04/024 688, WO 04/065 366, WO 04/080 962, WO 04/111 042, WO 05/044 791, WO 05/044 796, WO 05/048 710, WO 05/049 596, WO 05/066 125, WO 05/092897, WO 06/000355, WO 96/029799, WO 06/056281, WO 06/056282, WO 06/089633 and WO 07/048,545.

All active compounds present in the compositions according to the invention are already known and can be prepared by processes described in the prior art (see references mentioned above). Their activity is good; however, in particular at low application rates and concentrations, it is not always satisfactory. Furthermore, the compatibility of these compounds with crop plants is not always sufficient. Accordingly, there is a need for boosting the activity of the crop protection compositions comprising the compounds.

It has already been described in the literature that the activity of various active compounds may be enhanced by addition of ammonium salts. However, these are salts acting as detergents (for example WO 95/017817) or salts having relatively long alkyl and/or aryl substituents which act permeabilizingly or increase the solubility of the active compound (for example EP-A 0 453 086, EP-A 0 664 081, FR-A 2 600 494, U.S. Pat. No. 4,844,734, U.S. Pat. No. 5,462,912, U.S. Pat. No. 5,538,937, US-A 03/0224939, US-A 05/0009880, US-A 05/0096386). Furthermore, the prior art describes the effect only for certain active compounds and/or certain applications of the corresponding compositions. In other cases still, they are salts of sulphonic acids where the acids for their part act paralyzingly on insects (U.S. Pat. No. 2,842,476). An activity boost for example by ammonium sulphate has been described, for example, for the herbicides glyphosate and phosphinothricin (U.S. Pat. No. 6,645,914, EP-A2 0 036 106). This prior art neither discloses nor suggests a corresponding action for insecticides.

The use of ammonium sulphate as formulation auxiliary for certain active compounds and applications has also been described (WO 92/16108); however, in this publication it serves to stabilize the formulation, not to boost the activity.

Mixtures of ketoenols with fertilizers are known, too (WO 06/079079); however, these fertilizers are not specifically ammonium nitrate/urea solutions, and what is described is not an enhanced activity, but a way to solve problems with incompatibility.

The international patent application PCT/EP2006/011912 discloses enhanced activity for ketoenols with ammonium sulphate or ammonium sulphate in combination with a penetrant Ammonium sulphate is one of a plurality of customary fertilizer components. However, the present invention discloses activity boosts by other fertilizers not comprising any ammonium sulphate.

Totally surprisingly, it has now been found that the activity of insecticides and/or acaricides from the class of the phenyl-substituted tetramic acid derivatives can by enhanced considerably by addition of fertilizers to the solution applied or by incorporating fertilizers into a formulation comprising phenyl-substituted tetramic acid derivatives. Here, the activity boost is so high that the addition of further additives may be dispensed with. Since foliar fertilization of the crops is frequently carried out anyway, the use of the compositions according to the invention results in a markedly reduced environmental impact and in less work for the farmer.

Accordingly, the present invention also provides the use of fertilizers for boosting the activity of crop protection compositions comprising, as active compound, insecticidally and/or acaricidally active phenyl-substituted tetramic acid derivatives. The invention also provides compositions, both formulated active compounds and ready-to-use compositions (spray liquors) comprising insecticidally active phenyl-substituted tetramic acid derivatives and fertilizers. Finally, the invention furthermore provides the use of these compositions for controlling harmful insects and/or spider mites.

The tetramic acid derivatives are compounds of the formula (I)

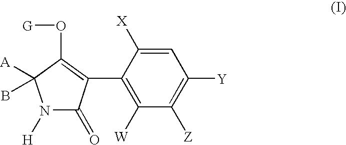

in which
X represents halogen, alkyl, alkoxy, haloalkyl, haloalkoxy or cyano,
W, Y and Z independently of each other represent hydrogen, halogen, alkyl, alkoxy, haloalkenyl, haloalkoxy or cyano,
A represents hydrogen, or represents in each case optionally halogen-substituted alkyl, alkoxyalkyl, saturated, optionally substituted cycloalkyl in which optionally at least one ring atom is replaced by a heteroatom,
B represents hydrogen or alkyl,
   or
A and B together with the carbon atom to which they are bonded represent a saturated or unsaturated, unsubstituted or substituted cycle which optionally contains at least one heteroatom,
G represents hydrogen (a) or one of the groups

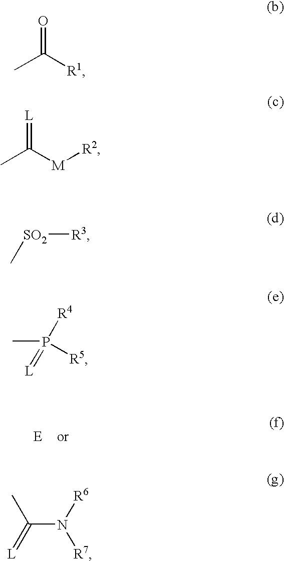

in which
E represents a metal ion or an ammonium ion,
L represents oxygen or sulphur,
M represents oxygen or sulphur,
R¹ represents in each case optionally halogen-substituted alkyl, alkenyl, alkoxyalkyl, alkylthioalkyl, polyalkoxyalkyl or optionally halogen-, alkyl- or alkoxy-substituted cycloalkyl which can be interrupted by at least one heteroatom, in each case optionally substituted phenyl, phenylalkyl, hetaryl, phenoxyalkyl or hetaryloxyalkyl,
   R² represents in each case optionally halogen-substituted alkyl, alkenyl, alkoxyalkyl, polyalkoxyalkyl or in each case optionally substituted cycloalkyl, phenyl or benzyl,
   R³ represents optionally halogen-substituted alkyl or optionally substituted phenyl,
   R⁴ and R⁵ independently of one another represent in each case optionally halogen-substituted alkyl, alkoxy, alkylamino, dialkylamino, alkylthio, alkenylthio, cycloalkylthio, or represent in each case optionally substituted phenyl, benzyl, phenoxy or phenylthio, and
   R⁶ and R⁷ independently of one another represent hydrogen, in each case optionally halogen-substituted alkyl, cycloalkyl, alkenyl, alkoxy, alkoxyalkyl, optionally substituted phenyl, optionally substituted benzyl or, together with the N atom to which they are bonded, represent an optionally substituted ring which is optionally interrupted by oxygen or sulphur,
      in the form of their isomer mixtures or their pure isomers.

Tetramic acid derivatives of the abovementioned formula (I) which can preferably be employed are those in which the radicals have the following meanings:
W preferably represents hydrogen, C₁-C₄-alkyl, C₁-C₄-alkoxy, chlorine, bromine or fluorine,
X preferably represents C₁-C₄-alkyl, C₁-C₄-alkoxy, C₁-C₄-haloalkyl, fluorine, chlorine or bromine,
Y and Z independently of one another preferably represent hydrogen, C₁-C₄-alkyl, halogen, C₁-C₄-alkoxy or C₁-C₄-haloalkyl,
A preferably represents hydrogen or in each case optionally halogen-substituted C₁-C₆-alkyl or C₃-C₈-cycloalkyl,
B preferably represents hydrogen, methyl or ethyl,
A,B and the carbon atom to which they are bonded preferably represent saturated C₃-C₆-cycloalkyl in which one ring member is optionally replaced by oxygen or sulphur and which is optionally monosubstituted or disubstituted by C₁-C₄-alkyl, trifluoromethyl or C₁-C₄-alkoxy,
G preferably represents hydrogen (a) or one of the groups

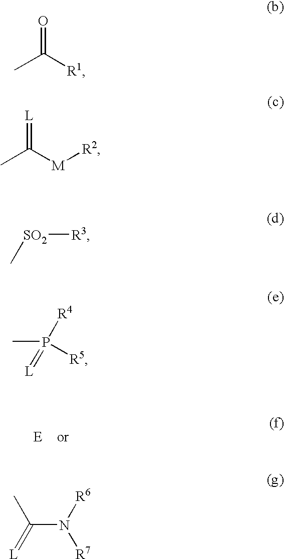

in particular (a), (b), (c) or (g)
in which
E represents a metal ion or an ammonium ion,
L represents oxygen or sulphur and
M represents oxygen or sulphur,
R¹ preferably represents in each case optionally halogen-substituted C₁-C₁₀-alkyl, C₂-C₁₀-alkenyl, C₁-C₄-alkoxy-C₁-C₄-alkyl, C₁-C₄-alkylthio-C₁-C₄-alkyl, or represents C₃-C₆-cycloalkyl which is optionally substituted by fluorine, chlorine, C₁-C₄-alkyl or C₁-C₂-alkoxy, or represents phenyl which is optionally substituted by fluorine, chlorine, bromine, cyano, nitro, C₁-C₄-alkyl, C₁-C₄-alkoxy, trifluoromethyl or trifluoromethoxy, or represents pyridyl or thienyl, each of which is optionally substituted by chlorine or methyl,
R² preferably represents in each case fluorine- or chlorine-substituted C₁-C₁₀-alkyl, C₂-C₁₀-alkenyl, C₁-C₄-alkoxy-C₂-C₄-alkyl, or represents optionally methyl- or methoxy-substituted C₅-C₆-cycloalkyl, or represents phenyl or benzyl, each of which is optionally substituted by fluorine, chlorine, bromine, cyano, nitro, C₁-C₄-alkyl, C₁-C₄-alkoxy, trifluoromethyl or trifluoromethoxy,
R³ preferably represents optionally fluorine-substituted C₁-C₄-alkyl, or represents phenyl which is optionally substituted by fluorine, chlorine, bromine, C₁-C₄-alkyl, C₁-C₄-alkoxy, trifluoromethyl, trifluoromethoxy, cyano or nitro,
R⁴ preferably represents in each case optionally fluorine- or chlorine-substituted C₁-C₄-alkyl, C₁-C₄-alkoxy, C₁-C₄-alkylamino, C₁-C₄-alkylthio or represents phenyl, phenoxy or phenylthio, each of which is optionally substituted by fluorine, chlorine, bromine, nitro, cyano, C₁-C₄-alkoxy, trifluoromethoxy, C₁-C₄-alkylthio, C₁-C₄-haloalkylthio, C₁-C₄-alkyl or trifluoromethyl,
R⁵ preferably represents C₁-C₄-alkoxy or C₁-C₄-thioalkyl,
R⁶ preferably represents C₁-C₆-alkyl, C₃-C₆-cycloalkyl, C₁-C₆-alkoxy, C₃-C₆-alkenyl or C₁-C₄-alkoxy-C₁-C₄-alkyl,
R⁷ preferably represents C₁-C₆-alkyl, C₃-C₆-alkenyl or C₁-C₄-alkoxy-C₁-C₄-alkyl,
R⁶ and R⁷ together preferably represent an optionally methyl- or ethyl-substituted C₃-C₆-alkylene radical in which one carbon atom is optionally replaced by oxygen or sulphur,
   in the form of their isomer mixtures or their pure isomers.

Tetramic acid derivatives of the abovementioned formula (I) which can especially preferably be employed are those in which the radicals have the following meanings:
W especially preferably represents hydrogen, methyl, ethyl, chlorine, bromine or methoxy,
X especially preferably represents chlorine, bromine, methyl, ethyl, propyl, i-propyl, methoxy, ethoxy or trifluoromethyl,
Y and Z especially preferably independently of one another represent hydrogen, fluorine, chlorine, bromine, methyl, ethyl, propyl, i-propyl, trifluoromethyl or methoxy,
A especially preferably represents methyl, ethyl, propyl, i-propyl, butyl, i-butyl, sec-butyl, tert-butyl, cyclopropyl, cyclopentyl or cyclohexyl,
B especially preferably represents hydrogen, methyl or ethyl,
   or
A, B and the carbon atom to which they are bonded especially preferably represent saturated C₆-cycloalkyl in which one ring member is optionally replaced by oxygen and which is optionally monosubstituted by methyl, ethyl, trifluoromethyl, methoxy, ethoxy, propoxy or butoxy,
G especially preferably represents hydrogen (a) or one of the groups

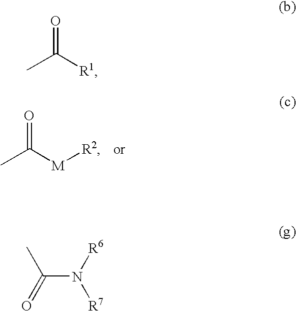

in which
M represents oxygen or sulphur,
R¹ especially preferably represents C₁-C₈-alkyl, C₂-C₄-alkenyl, methoxymethyl, ethoxymethyl, ethylthiomethyl, cyclopropyl, cyclopentyl or cyclohexyl, or represents phenyl which is optionally monosubstituted to disubstituted by fluorine, chlorine, bromine, cyano, nitro, methyl, ethyl, methoxy, trifluoromethyl or trifluoromethoxy, or represents pyridyl or thienyl, each of which is optionally substituted by chlorine or methyl,
R² especially preferably represents C₁-C₈-alkyl, C₂-C₄-alkenyl, methoxyethyl, ethoxyethyl, or represents phenyl or benzyl,
R⁶ and R⁷ independently of one another especially preferably represent methyl or ethyl or together represent a C₅-alkylene radical in which the C₃-methylene group is replaced by oxygen,
   in the form of their isomer mixtures or their pure isomers.

Tetramic acid derivatives of the abovementioned formula (I) which can very especially preferably be employed are those in which the radicals have the following meanings:
W very especially preferably represents hydrogen or methyl,
X very especially preferably represents chlorine, bromine or methyl,
Y and Z independently of one another very especially preferably represent hydrogen, chlorine, bromine or methyl,
A, B and the carbon atom to which they are bonded very especially preferably represent saturated C₆-cycloalkyl in which one ring member is optionally replaced by oxygen and which is optionally monosubstituted by methyl, trifluoromethyl, methoxy, ethoxy, propoxy or butoxy,
G very especially preferably represents hydrogen (a) or one of the groups

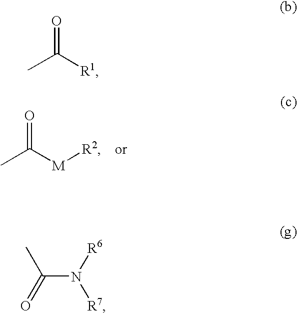

in which
M represents oxygen or sulphur,
R¹ very especially preferably represents C₁-C₈-alkyl, C₂-C₄-alkenyl, methoxymethyl, ethoxymethyl, ethylthiomethyl, cyclopropyl, cyclopentyl, cyclohexyl or represents phenyl which is optionally monosubstituted by fluorine, chlorine, bromine, methyl, methoxy, trifluoromethyl, trifluoromethoxy, cyano or nitro, or represents pyridyl or thienyl, each of which is optionally substituted by chlorine or methyl,
R² very especially preferably represents C₁-C₈-alkyl, C₂-C₄-alkenyl, methoxyethyl, ethoxyethyl, phenyl or benzyl,
R⁶ and R⁷ independently of one another very especially preferably represent methyl or ethyl or together represent a C₅-alkylene radical in which the C₃-methylene group is replaced by oxygen
   in the form of their isomer mixtures or their pure isomers.

Tetramic acid derivatives of the abovementioned formula (I) which can especially preferably be employed are those in which the radicals have the following meanings:

| | |
|---|---|
| | (I) |
| 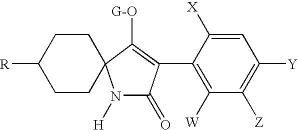 | |

| Ex- | | | | | | | |
|---|---|---|---|---|---|---|---|
| ample | | | | | | | |
| No. | W | X | Y | Z | R | G | M.p. ° C. |
| I-1 | H | Br | H | CH₃ | OCH₃ | CO-i-C₃H₇ | 122 |
| I-2 | H | Br | H | CH₃ | OCH₃ | CO₂—C₂H₅ | 140-142 |
| I-3 | H | CH₃ | H | CH₃ | OCH₃ | H | >220 |
| I-4 | H | CH₃ | H | CH₃ | OCH₃ | CO₂—C₂H₅ | 128 |
| I-5 | CH₃ | CH₃ | H | Br | OCH₃ | H | >220 |
| I-6 | CH₃ | CH₃ | H | Cl | OCH₃ | H | 219 |
| I-7 | H | Br | CH₃ | CH₃ | OCH₃ | CO-i-C₃H₇ | 217 |
| I-8 | H | CH₃ | Cl | CH₃ | OCH₃ | CO₂C₂H₅ | 162 |
| I-9 | CH₃ | CH₃ | CH₃ | CH₃ | OCH₃ | H | >220 |
| I-10 | CH₃ | CH₃ | H | Br | OC₂H₅ | CO-i-C₃H₇ | 212-214 |
| I-11 | H | CH₃ | CH₃ | CH₃ | OC₂H₅ | CO-n-C₃H₇ | 134 |
| I-12 | H | CH₃ | CH₃ | CH₃ | OC₂H₅ | CO-i-C₃H₇ | 108 |
| I-13 | H | CH₃ | CH₃ | CH₃ | OC₂H₅ | CO-c-C₃H₅ | 163 |

in the form of their cis/trans isomer mixtures or their pure cis isomers.

Compounds whose use must be emphasized are the compounds I-3 and I-4 as the cis isomers:

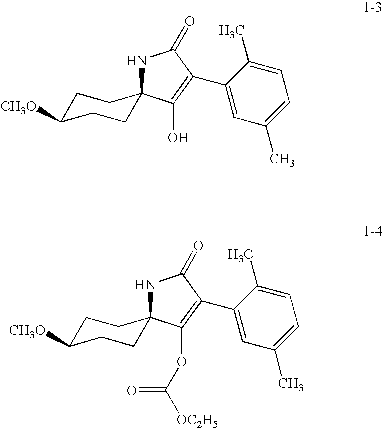

The compounds of the formula (I) are known compounds whose preparation is described in the patents/patent applications cited at the outset (see in particular WO 97/01535, WO 97/36868, WO 98/05 638 and WO 04/007448).

Suitable for the use according to the invention or the preparation of compositions according to the invention are, in principle, all fertilizers which can be used for foliar fertilization. Preference is given to fertilizers comprising urea. Particular preference is given to fertilizers comprising urea and at least one ammonium and/or phosphonium salt. Very particular preference is given to fertilizers comprising ammonium nitrate and urea. Especially preferred are fertilizers which comprise ammonium nitrate and urea and have a nitrogen content of 25 to 30 percent.

In general, the compositions according to the invention comprise, in addition to at least one tetramic acid derivative, urea in a concentration between 0.01 and 2 mol/l, preferably between 0.1 and 2 mol/l.

The tetramic acid derivatives can be applied in accordance with the invention on their own, but also in combination with other insecticidal, fungicidal and/or acaricidal active substances. The use according to the invention of the tetramic acid derivatives extends to a wide range of different animal pests. These include:

From the order of the Anoplura (Phthiraptera), for example Damalinia spp., Haematopinus spp., Linognathus spp., Pediculus spp., Trichodectes spp.

From the class of the Arachnida, for example Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example Dreissena spp.

From the order of the Chilopoda, for example Geophilus spp., Scutigera spp.

From the order of the Coleoptera, for example Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example Onychiurus armatus.

From the order of the Dermaptera, for example Forficula auricularia.

From the order of the Diplopoda, for example Blaniulus guttulatus.

From the order of the Diptera, for example Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp. Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example Anion spp., Biomphalaria spp., Bulinus spp., Deroceras spp., Galba spp., Lymnaea spp., Oncomelania spp., Succinea spp.

From the class of the Helminthes, for example Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

Protozoans such as Eimeria can also be controlled.

From the order of the Heteroptera, for example Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example Diprion spp., Hoplocampa spp., Lasius spp., Monomorium pharaonis, Vespa spp.

From the order of the Isopoda, for example Armadillidium vulgare, Oniscus asellus, Porcellio scaber.

From the order of the Isoptera, for example Reticulitermes spp., Odontotermes spp.

From the order of the Lepidoptera, for example Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example Ceratophyllus spp., Xenopsylla cheopis.

From the order of the Symphyla, for example Scutigerella immaculata.

From the order of the Thysanoptera, for example Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example Lepisma saccharina.

The plant-parasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

If appropriate, the compositions according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms).

In addition to the agrochemically active compounds already mentioned above, the compositions according to the invention may comprise, as mixing partners, further active compounds, such as insecticides, attractants, sterilizing agents, bactericides, acaricides, nematicides, fungicides, growth-regulating substances, herbicides, safeners, fertilizers or semiochemicals. Preferred according to the invention are systemic active compounds from the group of the insecticides and fungicides.

Particularly favourable mixing partners are, for example, the following:

### Fungicides:

### Inhibitors of Nucleic Acid Synthesis

benalaxyl, benalaxyl-M, bupirimate, chiralaxyl, clozylacon, dimethirimol, ethirimol, furalaxyl, hymexazol, mefenoxam, metalaxyl, metalaxyl-M, ofurace, oxadixyl, oxolinic acid

### Inhibitors of Mitosis and Cell Division

benomyl, carbendazim, diethofencarb, fuberidazole, thiabendazole, thiophanate-methyl

### Inhibitors of Respiratory Chain Complex II

boscalid, carboxin, fenfuram, flutolanil, furametpyr, furmecyclox, mepronil, oxycarboxin

### Inhibitors of Respiratory Chain Complex III

azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin

### Inhibitors of ATP Production

fentin acetate, fentin chloride, fentin hydroxide

### Inhibitors of Amino Acid Biosynthesis and Protein Biosynthesis

andoprim, cyprodinil, kasugamycin, kasugamycin hydrochloride hydrate, pyrimethanil

### Inhibitors of Signal Transduction

fludioxonil, quinoxyfen

### Inhibitors of Lipid and Membrane Synthesis

chlozolinate, iprodione, procymidone
ampropylfos, potassium-ampropylfos, edifenphos, etridiazole, iprobenfos (IBP), isoprothiolane, pyrazophos
biphenyl
iodocarb, propamocarb, propamocarb hydrochloride, propamocarb-fosetylate

### Inhibitors of Ergosterol Biosynthesis

azaconazole, bitertanol, bromuconazole, cyproconazole, diclobutrazole, difenoconazole, diniconazole, diniconazole-M, epoxiconazole, etaconazole, fenarimol, fenbuconazole, fluquinconazole, flurprimidole, flusilazole, flutriafol, furconazole, furconazole-cis, hexaconazole, imazalil, imazalil sulphate, imibenconazole, ipconazole, metconazole, myclobutanil, nuarimol, oxpoconazole, paclobutrazole, penconazole, pefurazoate, prochloraz, propiconazole, prothioconazole, pyrifenox, simeconazole, tebuconazole, tetraconazole, triadimefon, triadimenol, triflumizole, triforine, triticonazole, uniconazole, voriconazole, viniconazole,
aldimorph, dodemorph, dodemorph acetate, fenpropidin, fenpropimorph, spiroxamine, tridemorph,
naftifine, pyributicarb, terbinafine

### Inhibitors of Cell Wall Synthesis

benthiavalicarb, bialaphos, dimethomorph, flumorph, iprovalicarb, mandipropamid, polyoxins, polyoxorim

### Inhibitors of Melanin Biosynthesis

capropamid, diclocymet, fenoxanil, phthalid, pyroquilon, tricyclazole

### Resistance Inductors

acibenzolar-S-methyl, probenazole, tiadinil

### Further Fungicides

amibromdol, benthiazole, bethoxazin, capsimycin, carvone, chloropicrin, cufraneb, cymoxanil, dazomet, debacarb, diclomezine, difenzoquat, difenzoquat metilsulphate, dimetomorph, dithiofencarb, ferimzone, flumetover, flusulfamide, fluopicolide, fluoroimide, fosetyl-aluminium, fosetyl-calcium, fosetyl-sodium, hexachlorobenzene, 8-hydroxyquinoline sulphate, irumamycin, methasulfocarb, metrafenone, methyl isothiocyanate, mildiomycin, natamycin, nickel dimethyl dithiocarbamate, octhilinone, oxamocarb, oxyfenthiin, pentachlorophenol and salts, 2-phenylphenol and salts, piperalin, propanosine-sodium, pyribencarb, pyrroInitrin, quintozene, tecloftalam, tecnazene, trichlamide, valiphenal, zarilamid,
2-(2-{[6-(3-chloro-2-methylphenoxy)-5-fluoropyrimidin-4-yl]oxy}phenyl)-2-(methoxyimino)-N-methylacetamide,
2-[[[[1-[3-(1-fluoro-2-phenylethyl)oxy]phenyl]ethylidene]amino]oxy]methyl]-alpha-(methoxyimino)-N-methyl-alpha-benzacetamide,
cis-1-(4-chlorophenyl)-2-(1H-1,2,4-triazol-1-yl)cycloheptanol,
1-[(4-methoxyphenoxy)methyl]-2,2-dimethylpropyl-1H-imidazole-1-carboxylic acid,
2,3,5,6-tetrachloro-4-(methylsulphonyl)pyridine,
2-butoxy-6-iodo-3-propylbenzopyranon-4-one,
2-chloro-N-(2,3-dihydro-1,1,3-trimethyl-1H-inden-4-yl)-3-pyridinecarboxamide,
3,4,5-trichloro-2,6-pyridinedicarbonitrile,
3,4-dichloro-N-(2-cyanophenyl)isothiazole-5-carboxamide (isotianil)
3-[5-(4-chlorophenyl)-2,3-dimethylisoxazolidin-3-yl]pyridine,
5-chloro-6-(2,4,6-trifluorophenyl)-N-[(1R)-1,2,2-trimethylpropyl][1,2,4]triazolo[1,5-a]pyrimidine-7-amine,
5-chloro-7-(4-methylpiperidin-1-yl)-6-(2,4,6-trifluorophenyl)[1,2,4]triazolo[1,5-a]pyrimidine,
5-chloro-N-[(1R)-1,2-dimethylpropyl]-6-(2,4,6-trifluorophenyl) [1,2,4]triazolo[1,5-a]pyrimidine-7-amine,
methyl 2-[[[cyclopropyl[(4-methoxyphenyl)imino]methyl]thio]methyl]-alpha-(methoxymethylene) benzacetate,
methyl 1-(2,3-dihydro-2,2-dimethyl-1H-inden-1-yl)-1H-imidazole-5-carboxylate,
N-(3′,4′-dichloro-5-fluorobiphenyl-2-yl)-3-(difluoromethyl)-1-methyl-1H-pyrazole-4-carboxamide,
N-(3-ethyl-3,5,5-trimethylcyclohexyl)-3-formylamino-2-hydroxy benzamide,
N-(4-chloro-2-nitrophenyl)-N-ethyl-4-methylbenzenesulphonamide,
N-(4-chlorobenzyl)-3-[3-methoxy-4-(prop-2-yn-1-yloxy)phenyl]propanamide,
N-[(4-chlorophenyl)(cyano)methyl]-3-[3-methoxy-4-(prop-2-yn-1-yloxy)phenyl]propanamide,
N-(5-bromo-3-chloropyridin-2-yl)methyl-2,4-dichloronicotinamide,
N-[1-(5-bromo-3-chloropyridin-2-yl)ethyl]-2,4-dichloronicotinamide,
(2S)—N-[2-[4-[[3-(4-chlorophenyl)-2-propynyl]oxy]-3-methoxyphenyl]ethyl]-3-methyl-2-[(methylsulphonyl)amino]butanamide,
N-{(Z)-[(cyclopropylmethoxy)imino][6-(difluoromethoxy)-2,3-difluorophenyl]methyl}-2-benzacetamide,
N-{2-[1,1′-bi(cyclopropyl)-2-yl]phenyl}-3-(difluoromethyl)-1-methyl-1H-pyrazole-4-carboxamide,
N-{2-[3-chloro-5-(trifluoromethyl)pyridin-2-yl]ethyl}-2-(trifluoromethyl)benzamide,
N-ethyl-N-methyl-N′-{2-methyl-5-(trifluoromethyl)-4-[3-(trimethylsilyl)propoxy]phenyl}imidoformamide,
O-[1-[(4-methoxyphenoxy)methyl]-2,2-dimethylpropyl]-1H-imidazole-1-carbothioic acid,
2-amino-4-methyl-N-phenyl-5-thiazolecarboxamide,
2,4-dihydro-5-methoxy-2-methyl-4-[[[[143-(trifluoromethyl)phenyl]ethylidene]amino]oxy]methyl]phenyl]-3H-1,2,4-triazol-3-one (CAS No. 185336-79-2) and
N-(6-methoxy-3-pyridinyl)cyclopropane carboxamide.

Fungicides which may preferably be used according to the invention are

etridiazole, fosetyl-aluminium, propamocarb hydrochloride, metalaxyl, metalaxyl-M, benalaxyl-M, azoxystrobin, dimetomorph, pyrimethanil, carbendazim, dithiofencarb, thiophanate-methyl, prochloraz, boscalid, trifloxystrobin, fluoxastrobin, iprodione, propamocarb fosetylate, prothioconazole, triticonazole, fluquinconazole, triadimenol, iprovalicarb, fluopicolide, N-{2-[1,1′-bi(cyclopropyl)-2-yl]phenyl}-3-(difluoromethyl)-1-methyl-1H-pyrazole-4-carboxamide, N-[2-(1,3-dimethylbutyl)phenyl]-5-fluoro-1,3-dimethyl-1H-pyrazole-4-carboxamide, N-(3′,4′-dichloro-5-fluoro-1,1′-biphenyl-2-yl)-3-(difluoromethyl)-1-methyl-1H-pyrazole-4-carboxamide, N-{2-[3-chloro-5-(trifluoromethyl)-2-pyridinyl]ethyl}-2-trifluoromethylbenzamide, 5-chloro-6-(2,4,6-trifluorophenyl)-N-[(1R)-1,2,2-trimethylpropyl][1,2,4]triazolo[1,5-a]pyrimidine-7-amine, 5-chloro-N-[(1R)-1,2-dimethylpropyl]-6-(2,4,6-trifluorophenyl)[1,2,4]triazolo[1,5-a]pyrimidine-7-amine and 5-chloro-7-(4-methylpiperidin-1-yl)-6-(2,4,6-trifluorophenyl)[1,2,4]triazolo[1,5-a]pyrimidine,

### Bactericides:

bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

### Insecticides/Acaricides/Nematicides:

### Acetylcholine Esterase (AChE) Inhibitors

carbamates,
for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulphan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate
organophosphates,
for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphone, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulphothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulphotep, sulprofos, tebupirimfos, temephos, terbufos, tetrachlorvinphos, thiometon, triazophos, triclorfon, vamidothion

### Sodium Channel Modulators/Voltage-Dependent Sodium Channel Blockers

pyrethroids,
for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)

### DDT

oxadiazines,
   for example indoxacarb
semicarbazones,
   for example metaflumizone (BAS3201)

### Acetylcholine Receptor Agonists/Antagonists

chloronicotinyls,
   for example acetamiprid, clothianidin, dinotefuran, imidacloprid, nitenpyram, nithiazine, thiacloprid, thiamethoxam
nicotine, bensultap, cartap

### Acetylcholine Receptor Modulators

spinosyns,
for example spinosad

### GABA-Controlled Chloride Channel Antagonists

organochlorines,
   for example camphechlor, chlordane, endosulphan, gamma-HCH, HCH, heptachlor, lindane, methoxychlor
fiprols,
   for example acetoprole, ethiprole, fipronil, pyrafluprole, pyriprole, vaniliprole

### Chloride Channel Activators

mectins,
for example abamectin, emamectin, emamectin-benzoate, ivermectin, lepimectin, milbemycin

### Juvenile Hormone Mimetics,

for example diofenolan, epofenonane, fenoxycarb, hydroprene, kinoprene, methoprene, pyriproxifen, triprene

### Ecdysone Agonists/Disruptors

diacylhydrazines,
for example chromafenozide, halofenozide, methoxyfenozide, tebufenozide

### Chitin Biosynthesis Inhibitors

benzoylureas,
   for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron
buprofezin
cyromazine

### Oxidative Phosphorylation Inhibitors, ATP Disruptors

diafenthiuron
organotin compounds,
   for example azocyclotin, cyhexatin, fenbutatin-oxide

### Oxidative Phosphorylation Decouplers Acting by Interrupting the H-Proton Gradient

pyrroles,
   for example chlorfenapyr
dinitrophenols,
   for example binapacyrl, dinobuton, dinocap, DNOC, meptyldinocap

### Site-I Electron Transport Inhibitors

METI's,
   for example fenazaquin, fenpyroximate, pyrimidifen, pyridaben, tebufenpyrad, tolfenpyrad
hydramethylnon
dicofol

### Site-II Electron Transport Inhibitors

rotenone

### Site-III Electron Transport Inhibitors

acequinocyl, fluacrypyrim

### Microbial Disruptors of the Insect Gut Membrane

Bacillus thuringiensis strains

### Lipid Synthesis Inhibitors

tetronic acids,
for example spirodiclofen, spiromesifen

### Carboxamides,

for example flonicamid

### Octopaminergic Agonists,

for example amitraz

### Inhibitors of Magnesium-Stimulated ATPase,

propargite
nereistoxin analogues,
for example thiocyclam hydrogen oxalate, thiosultap-sodium

### Ryanodine Receptor Agonists,

benzodicarboxamides,
for example flubendiamide
anthranilamides,
for example rynaxypyr (3-bromo-N-{4-chloro-2-methyl-6-[(methylamino)carbonyl]phenyl}-1-(3-chloropyridin-2-yl)-1H-pyrazole-5-carboxamide)

### Biologicals, Hormones or Pheromones

azadirachtin, Bacillus spec., Beauveria spec., codlemone, Metarrhizium spec., Paecilomyces spec., thuringiensin, Verticillium spec.
Active Compounds with Unknown or Unspecific Mechanisms of Action
fumigants,
   for example aluminium phosphide, methyl bromide, sulphuryl fluoride
antifeedants,
   for example cryolite, flonicamid, pymetrozine
mite growth inhibitors,
   for example clofentezine, etoxazole, hexythiazox
amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulphluramid, tetradifon, tetrasul, triarathene, verbutin

A mixture with other known active compounds, such as herbicides, fertilizers, growth regulators, safeners, semiochemicals, or else with agents for improving the plant properties, is also possible.

When used as insecticides, the compositions according to the invention can furthermore be present in their commercially available formulations and in the use forms, prepared from these formulations, as a mixture with synergistic agents. Synergistic agents are compounds which increase the action of the active compounds contained in the compositions according to the invention, without it being necessary for the synergistic agent added to be active itself.

When used as insecticides, the compositions according to the invention can furthermore be present in their commercially available formulations and in the use forms, prepared from these formulations, as a mixture with inhibitors which reduce degradation of the agrochemical active compound contained after use in the environment of the plant, on the surface of parts of plants or in plant tissues.

The active compound content of the use forms prepared from the commercially available formulations can vary within wide limits. The active compound concentration of the use forms can be from 0.00000001 to 95% by weight of active compound, preferably between 0.00001 and 1% by weight.

The compositions are employed in a customary manner appropriate for the use forms.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the compositions is carried out directly or by allowing the compounds to act on the surroundings, habitat or storage space by the customary treatment methods, for example by immersion, spraying, evaporation, fogging, scattering, painting on, injection and, in the case of propagation material, in particular in the case of seeds, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

Preference is given to foliar application by spraying.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particular advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized are in particular increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compositions according to the invention. The preferred ranges stated above for the compositions also apply to the treatment of these plants. Particular emphasis is given to the treatment of plants with the compositions specifically mentioned in the present text.

### USE EXAMPLE A

In three replications, plots of a size of about 66 m² with cotton of the cultivar FM966LL are treated against Aphis gossypii. Here, the active compound I-4 (as 1500D formulation) is applied with a sprayer (High Clearance Sprayer, High Boy) at the stated application rates. The amount of water applied is about 150 l/ha. An adjuvant and/or a fertilizer (UAN=ammonium nitrate/urea solution with 28% by weight of nitrogen) are added to the spray liquor at the stated concentration. Here, it becomes evident that the composition according to the invention is equivalent to a composition comprising an adjuvant known to be efficient.

| | Application rate | Effect (% Abbott) | | |
|---|---|---|---|---|
| | g of a.i./ha or % | After | After | After |
| Active compound | (v/v) of a.i./ha* | 2 days | 5 days | 9 days |
| I-4 | 44 | 66.7 | 65.8 | 13.9 |
| I-4 + Dyne-amic ® | 44 + 0.455* | 78.9 | 89.5 | 50 |
| I-4 + UAN | 44 + 0.7* | 85 | 89.8 | 47.2 |
| I-4 + Dyne-amic ® + | 44 + 0.455* + | 83.3 | 93.6 | 72.2 |
| UAN | 0.7* | | | |

### EXAMPLE B-1

To prepare a suitable product solution, 1 part by weight of formulated goods (for example an oil suspension OD 150 prepared according to known methods) is mixed with water to give the desired concentration. The desired fertilizer or penetrant is added in a defined amount and mixed in well.

Cotton plants (Gossypium herbaceum) which are heavily infested by the cotton aphids (Aphis gossypii) are treated by spraying with the product solution at the desired concentration.

After the desired period of time, the kill in % is determined. Here, 100% means that all aphids have been killed; 0% means that none of the aphids have been killed.

Table B-1 shows the markedly enhanced activity of the mixture compared to the active compound applied individually and an activity which, compared to the good penetrant, is almost equivalent.

TABLE B-1

| | | Kill |
|---|---|---|
| Active compound | Concentration | in % after 7^{d} |
| Compound I-4 | 4.8 g ai/ha | 90 |
| Control | 0.96 g ai/ha | 25 |
| AHL added | | |
| Compound I-4 | 4.8 g ai/ha | 95 |
| AHL | 0.96 g ai/ha | 65 |
| | 12.5 g/l | |
| NH₄NO₃ added | | |
| Compound I-4 | 4.8 g ai/ha | 95 |
| NH₄NO₃ | 0.96 g ai/ha | 55 |
| | 12.5 g/l | |
| RME added | | |
| Compound I-4 | 4.8 g ai/ha | 95 |
| RME (as EW 500) | 0.96 g ai/ha | 80 |
| | 1 g ai/l | |

RME = oilseed rape methyl ester
ai/ha = active ingredient/hectare

### USE EXAMPLE C

In three replications, plots of a size of about 36 m² with cotton of the cultivar DP555BR are treated against Bemisia tabaci. Here, the active compound I-4 (as 1500D formulation) is applied with a sprayer (6.4 km/h) at the stated application rates. The amount of water applied is about 142 l/ha. An adjuvant and/or a fertilizer (UAN=ammonium nitrate/urea solution with 28% by weight of nitrogen) are added to the spray liquor at the stated concentration. Two applications are carried out at an interval of 16 days. Evaluation is carried out 7, 14 and 20 days after the first application by scoring the effect (% Abbott) on the nymphs on the leaves. Here, it becomes evident that the composition according to the invention is equivalent to a composition comprising an adjuvant known to be efficient.

| | Application rate | Effect (% Abbott) | | |
|---|---|---|---|---|
| | g of a.i./ha or % | After | After | After |
| Active compound | (v/v) of a.i./ha* | 7 days | 14 days | 20 days |
| I-4 + Dyne-amic ® | 66 + 0.455* | 92 | 64 | 89 |
| I-4 + UAN | 66 + 0.7* | 94 | 71 | 84 |
| I-4 + Dyne-amic ® + | 66 + 0.455* + | 92 | 91 | 91 |
| UAN | 0.7* | | | |

1. A method for boosting the activity of a crop protection composition comprising using as a fertilizer at least one compound of the formula (I) 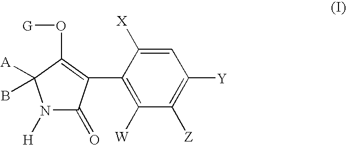
in which
X represents halogen, alkyl, alkoxy, haloalkyl, haloalkoxy or cyano,
W, Y and Z independently of each other represent hydrogen, halogen, alkyl, alkoxy, haloalkenyl, haloalkoxy or cyano,
A represents hydrogen, or represents in each case optionally halogen-substituted alkyl, alkoxyalkyl, saturated, optionally substituted cycloalkyl in which optionally at least one ring atom is replaced by a heteroatom,
B represents hydrogen or alkyl,
or
A and B together with the carbon atom to which they are bonded represent a saturated or unsaturated, unsubstituted or substituted cycle which optionally contains at least one heteroatom,
G represents hydrogen (a) or one of the groups 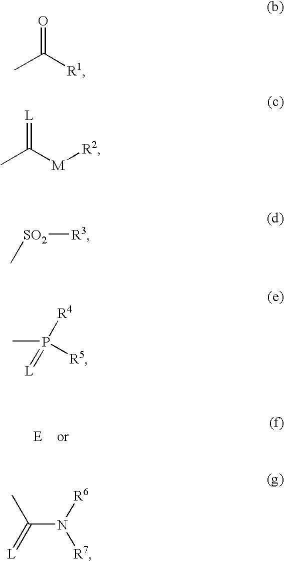
in which
E represents a metal ion or an ammonium ion,
L represents oxygen or sulphur,
M represents oxygen or sulphur,
R¹ represents in each case optionally halogen-substituted alkyl, alkenyl, alkoxyalkyl, alkylthioalkyl, polyalkoxyalkyl or optionally halogen-, alkyl- or alkoxy-substituted cycloalkyl which can be interrupted by at least one heteroatom, in each case optionally substituted phenyl, phenylalkyl, hetaryl, phenoxyalkyl or hetaryloxyalkyl,
R² represents in each case optionally halogen-substituted alkyl, alkenyl, alkoxyalkyl, polyalkoxyalkyl or in each case optionally substituted cycloalkyl, phenyl or benzyl,
R³ represents optionally halogen-substituted alkyl or optionally substituted phenyl,
R⁴ and R⁵ independently of one another represent in each case optionally halogen-substituted alkyl, alkoxy, alkylamino, dialkylamino, alkylthio, alkenylthio, cycloalkylthio, or represent in each case optionally substituted phenyl, benzyl, phenoxy or phenylthio, and
R⁶ and R⁷ independently of one another represent hydrogen, in each case optionally halogen-substituted alkyl, cycloalkyl, alkenyl, alkoxy, alkoxyalkyl, optionally substituted phenyl, optionally substituted benzyl or, together with the N atom to which they are bonded, represent an optionally substituted ring which is optionally interrupted by oxygen or sulphur
as an isomer mixture and/or as a pure isomer.

2. A method according to claim 1, wherein the fertilizer comprises ammonium nitrate and/or urea.

3. A method according to claim 1 wherein the fertilizer is ammonium sulphate-free.

4. A composition, comprising
ammonium nitrate and/or urea and
at least one active compound of the formula (I) 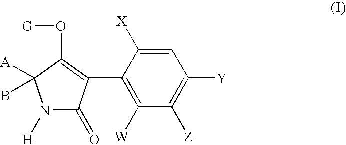
in which
X represents halogen, alkyl, alkoxy, haloalkyl, haloalkoxy or cyano,
W, Y and Z independently of each other represent hydrogen, halogen, alkyl, alkoxy, haloalkenyl, haloalkoxy or cyano,
A represents hydrogen, or represents in each case optionally halogen-substituted alkyl, alkoxyalkyl, saturated, optionally substituted cycloalkyl in which optionally at least one ring atom is replaced by a heteroatom,
B represents hydrogen or alkyl,
or
A and B together with the carbon atom to which they are bonded represent a saturated or unsaturated, unsubstituted or substituted cycle which optionally contains at least one heteroatom,
G represents hydrogen (a) or one of the groups 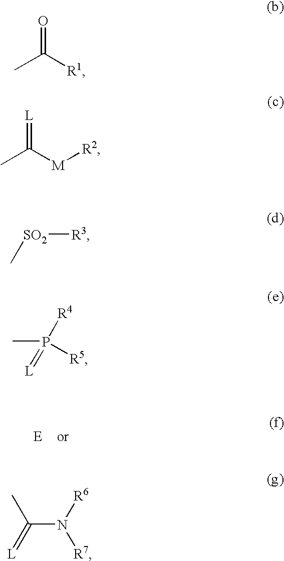
in which
E represents a metal ion or an ammonium ion,
L represents oxygen or sulphur,
M represents oxygen or sulphur,
R¹ represents in each case optionally halogen-substituted alkyl, alkenyl, alkoxyalkyl, alkylthioalkyl, polyalkoxyalkyl or optionally halogen-, alkyl- or alkoxy-substituted cycloalkyl which can be interrupted by at least one heteroatom, in each case optionally substituted phenyl, phenylalkyl, hetaryl, phenoxyalkyl or hetaryloxyalkyl,
R² represents in each case optionally halogen-substituted alkyl, alkenyl, alkoxyalkyl, polyalkoxyalkyl or in each case optionally substituted cycloalkyl, phenyl or benzyl,
R³ represents optionally halogen-substituted alkyl or optionally substituted phenyl,
R⁴ and R⁵ independently of one another represent in each case optionally halogen-substituted alkyl, alkoxy, alkylamino, dialkylamino, alkylthio, alkenylthio, cycloalkylthio, or represent in each case optionally substituted phenyl, benzyl, phenoxy or phenylthio, and
R⁶ and R⁷ independently of one another represent hydrogen, in each case optionally halogen-substituted alkyl, cycloalkyl, alkenyl, alkoxy, alkoxyalkyl, optionally substituted phenyl, optionally substituted benzyl or, together with the N atom to which they are bonded, represent an optionally substituted ring which is optionally interrupted by oxygen or sulphur
as an isomer mixture and/or as a pure isomer.

5. Composition according to claim 4, comprising ammonium nitrate and urea.

6. Composition according to claim 4, comprising at least one of the following compounds:

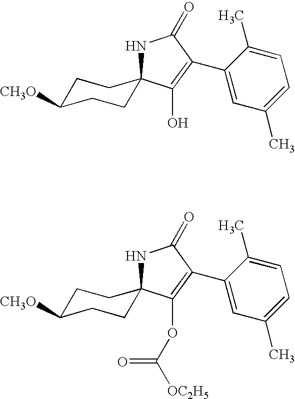

7. Method for boosting the activity of a crop protection composition according to claim 1, wherein said fertilizer is added to a concentrated or ready-to-use solution.

8. Composition according to claim 5, comprising at least one of the following compounds:

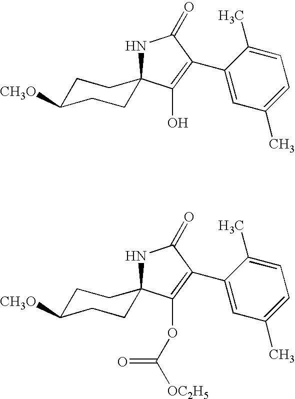

9. Method for boosting the activity of a crop protection composition according to claim 2, wherein said fertilizer is added to a concentrated or a ready to use solution.

10. Method for boosting the activity of a crop protection composition according to claim 3, wherein said fertilizer is added to a concentrated or a ready to use solution.

11. A method according to claim 2 wherein the fertilizer is ammonium sulphate-free.

12. A composition of claim 4 which is ammonium sulphate-free.

13. A composition of claim 5, which is ammonium sulphate-free.

14. A composition of claim 6, which is ammonium sulphate-free.

15. A composition of claim 8, which is ammonium sulphate-free.
